(19) United States
(12) Patent Application Publication
(10) Pub. No.: US 2009/0287061 A1
(43) Pub. Date: Nov. 19, 2009

(54) SURGICAL ACCESS DEVICE FOR MINIMALLY INVASIVE SURGERY

(75) Inventors: FRANK FEIGENBAUM, Mission Hills, KS (US); BOBBY TAY, San Raphael, CA (US); WESLEY GRIFFITT, Kansas City, MO (US)

Correspondence Address: SHOOK, HARDY & BACON LLP;INTELLECTUAL PROPERTY DEPARTMENT, 2555 GRAND BLVD, KANSAS CITY, MO, 64108-2613, US

(73) Assignee: GFT TECHNOLOGIES, San Raphael, CA (US)

(21) Appl. No.: 12/243,550

(22) Filed: Oct. 1, 2008

Related U.S. Application Data
(60) Provisional application No. 60/976,627, filed on Oct. 1, 2007.

(51) Int. Cl. A61B 1/32

(52) U.S. Cl. 600/204

(57) ABSTRACT

A method for employing a device to enable access at a surgical location adjacent to a spine, thereby allowing one or more surgical instruments to perform a minimally invasive spine operation, is provided herein. The device for providing access to the surgical location includes an elongate body that includes an outer tube and an inner tube. The inner tube is moveably received (e.g., via a worm drive) within the outer tube. Accordingly, the inner tube is selectively extensible and retractable relative to the outer tube in a telescopic manner. The extension and retraction capabilities of the device facilitate proper placement of the device within the surgical location. In addition, both the inner tube and the outer tube include inner surfaces. These inner surfaces define a passage extending through the elongate body. In operation, the passage allows for inserting surgical instruments through the device into the surgical location.

### CROSS-REFERENCE TO RELATED APPLICATION

This application claims the benefit of U.S. Provisional Application No. 60/976,627, filed Oct. 1, 2007, and is expressly incorporated by reference herein in its entirety.

### BACKGROUND OF THE INVENTION

1. Field of the Invention

This application relates to surgical systems and assemblies that include an access device for minimally invasive surgery, and in particular relates to devices that provide access to a surgical location, e.g., adjacent the spine, for one or more instruments to perform a procedure at the surgical location.

2. Description of the Related Art

Minimally invasive spine surgery allows surgeons access to the posterior spine through a process that is less traumatic to patients than traditional spine surgery. Instead of retracting the tissues and muscles surrounding the area of interest, only a small, localized amount is displaced. Therefore, less damage occurs to tissue and muscle, less scar tissue, and less recovery time. This surgery allows patients undergoing crucial procedures to have full mobility and recovery quickly after the procedure.

Typically, in a minimally invasive surgery procedure, a simple tube system is used to gain access to the posterior spine. A small incision is made in the back of the patient and small dilators of increasing size are inserted into the incision. A tube is inserted over the dilators and clamped down. Then, the dilators are removed and the surgeon operates through the tube.

### SUMMARY OF THE INVENTION

In one embodiment, a device is provided for accessing a surgical location within a patient. The device comprises an elongate body having an outer tube and an inner tube, with each of the tubes having a inner surface. The inner surfaces define a passage extending through the elongate body and through which surgical instruments may be inserted to the surgical location. The elongate body is capable of having a configuration when inserted within the patient wherein the elongate body is selectively extensible and retractable such that the distal end of the inner tube moves from a first location to a second location that is farther removed from the proximal end of the outer tube of the elongate body. The proximal portion of the device may comprise a means for extending the distal end of the body.

In one embodiment, the access device may further provide enhanced access to a spinal location within a patient by providing a window or opening in the inner tube that extends around a portion of the periphery of the inner tube adjacent a distal end of the inner tube. The access device further includes an extensible shutter for selectively covering or uncovering the window. The shutter may extend past the distal portion of the inner tube.

### BRIEF DESCRIPTION OF THE DRAWINGS

Further objects, features and advantages of the invention will become apparent from the following detailed description taken in conjunction with the accompanying figures showing illustrative embodiments of the invention, in which:

FIG. 1 is an exploded view of an access device of the present invention for treating the spine of a patient.

FIG. 2 is a perspective view of the access device in a retracted profile configuration.

FIG. 3 is a cross sectional view of the access device of FIG. 2 in an extended configuration.

FIG. 4 is a partial side elevational view of the access device of FIG. 3 showing the opening of the inner tube and the selectively movable shutter.

FIG. 5 is a partial perspective view of one embodiment of the access device with the shutter in a fully extended position.

Throughout the figures, the same reference numerals and characters, unless otherwise stated, are used to denote like features, elements, components or portions of the illustrated embodiments. Moreover, while the subject matter of this application will now be described in detail with reference to the figures, it is done so in connection with the illustrative embodiments. It is intended that changes and modifications can be made to the described embodiments without departing from the true scope and spirit of the subject invention as defined by the appended claims.

### DETAILED DESCRIPTION OF THE PREFERRED EMBODIMENTS

As should be understood in view of the following detailed description, this application is primarily directed to, though not necessarily limited to, an apparatus and method for treating the spine of a patient through an access device. More particularly, the system described below provides access to surgical locations at or near the spine and provides a tool useful in performing treatment of the spine.

The embodiment of the apparatus described herein will be discussed in terms of a minimally invasive procedure and apparatus, e.g., of endoscopic apparatuses and procedures. However, many aspects of the present invention may find use in conventional, open, and mini-open procedures. As used herein, the term “proximal,” as is traditional, refers to the end portion of the apparatus that is closest to the operator, while the term “distal” refers to the end portion that is farthest from the operator.

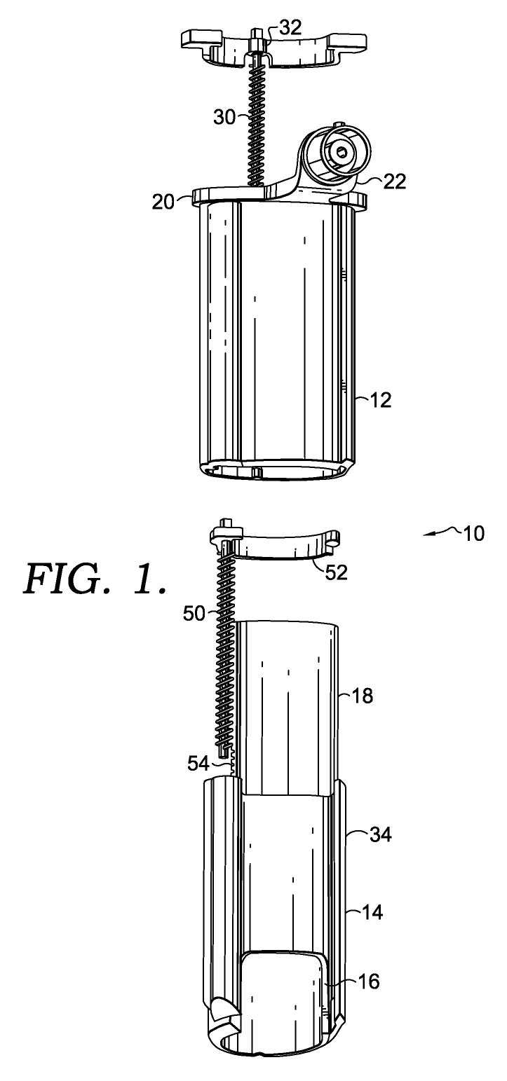

FIG. 1 shows one embodiment of a surgical access device 10 that can facilitate various surgical procedures. The access device 10 provides an internal passage for surgical instruments to be inserted through the skin and muscle tissue of a patient to the surgical site. The term “access device” is used in its ordinary sense to mean a device that can provide access and is a broad term and it includes structures having an elongate dimension and defining a passage, e.g., a cannula or a conduit. The access device is configured to be inserted through the skin of the patient to provide access during a surgical procedure to a surgical location within a patient, e.g., a spinal location. The term “surgical location” is used in its ordinary sense (i.e. a location where a surgical procedure is performed) and is a broad term and it includes locations subject to or affected by a surgery. The term “spinal location” is used in its ordinary sense (i.e. a location at or near a spine) and is a broad term and it includes locations adjacent to or associated with a spine that may be sites for surgical spinal procedures.

The access device is operably configured to ensure durability while also striving to minimize outer tube diameter and optimize inner working space. The access device comprises an elongate body defining a passageway. The elongate body includes an outer tube 12 and an inner tube 14 operably configured to be moveably received by the outer tube such that the inner tube is selectively extensible and retractable relative to the outer tube. The inner tube further includes an opening 16 formed around a portion of the periphery of the inner tube and a selectively extensible shutter 18 for covering and uncovering at least a portion of the opening.

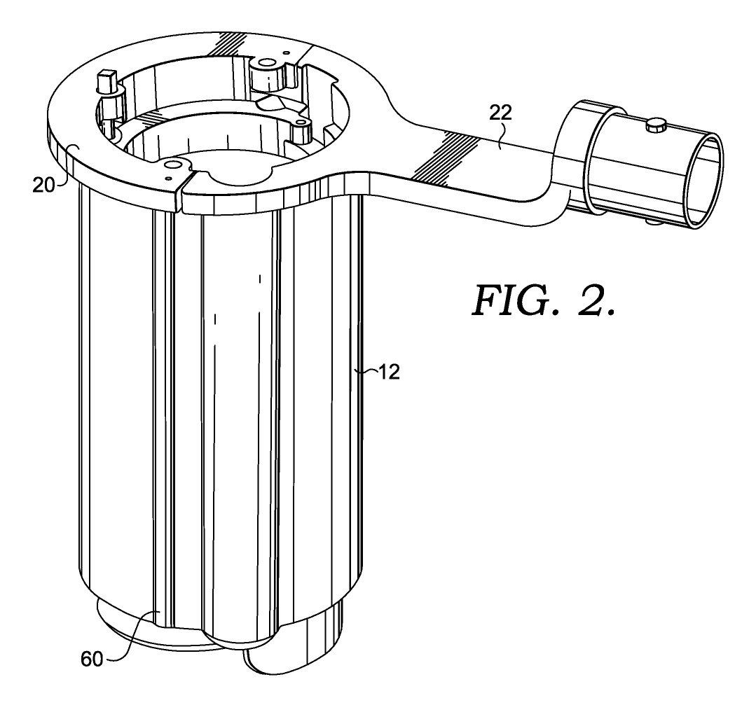

The outer tube 12 is approximately 50 mm long and, as best seen in FIG. 2, includes a flange 20 extending approximately 3 mm from the outer periphery of the outer tube. The flange 20 assists in constraining the patient's skin and prevents the tube from falling into the patient. The outer tube 12 further includes an approximately 25 mm in length arm 22 that extends outwardly from the flange 20 and connects to the snake arm. This arm 22 stabilizes the access apparatus 10 by rigidly fixing it in reference to the operating table (not shown).

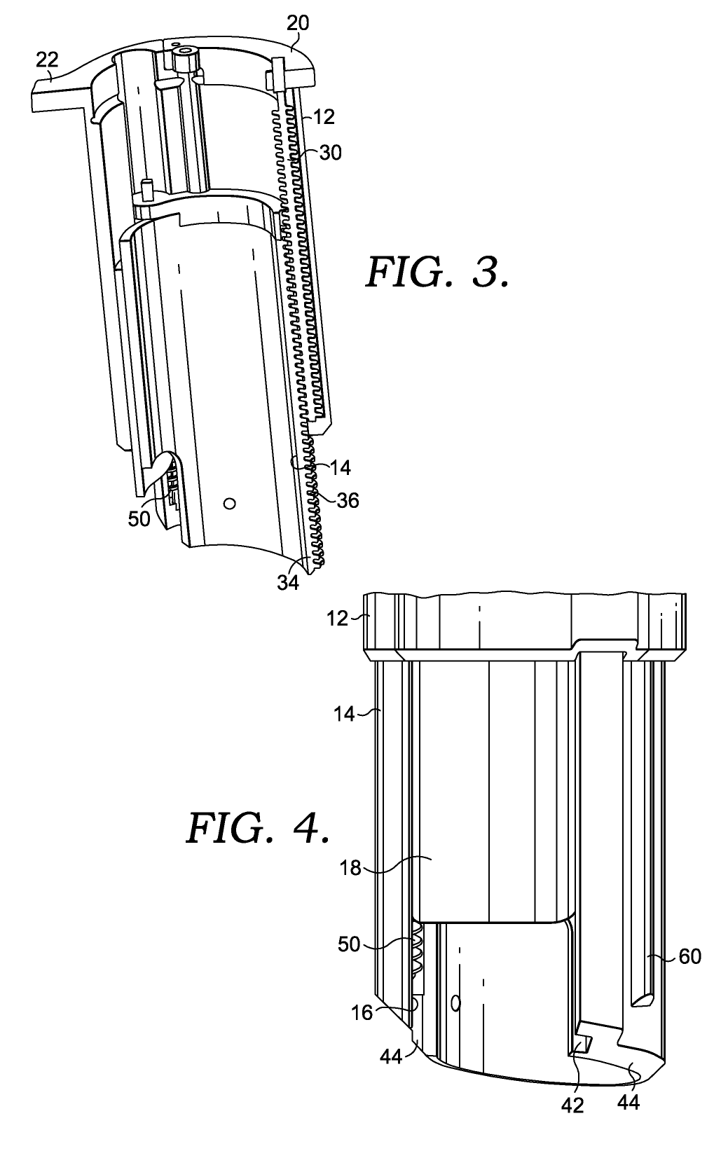

As shown in FIG. 3, the inner tube 14 is slidably received internally of the outer tube 12. The inner tube 14 is selectively extensible and retractable relative to the outer tube 16 to facilitate the proper placement of the tube in the surgical site. It is necessary to minimize the tube profile by using the shortest access device in situ to optimize working space for the surgeon. The elongate body 10 may be adjusted to the proper length within a range of approximately 55 mm to approximately 90 mm. As shown in FIGS. 1 and 3, a first worm drive 30 is mounted to a first snap ring 32 that is received on the upper or proximal edge of the outer tube 12. The first worm drive 30 is preferably approximately 46 mm in length and mates with teeth 36 formed on the exterior surface 34 of the inner tube 14. As the worm drive is rotationally twisted in one direction at an upper surface of the outer tube, the inner tube is extended outwardly from the bottom edge of the outer tube in a telescoping manner. Likewise, when the worm drive is rotationally twisted in the opposite directions, the inner tube retracts into the outer tube in a telescoping manner. A graduated scale 38 may be located on the outside of the inner tube for coarse adjustments prior to insertion.

As shown in FIG. 4. the inner tube 14 includes a distal peripheral edge 42 that may be formed with an approximately 12° medial bevel thereon. This allows the inner tube 14 to sit tightly against the vertebral lamina thereby minimizing lower tissue creep. Bevels on the tube edges and smooth radii around the tube ease provide a reduced profile for initial percutaneous insertion into the patient and subsequent rotation of the access device in the patient.

The inner tube 14 may be configured with the medial opening 16. Preferably, the opening or window 16 extends approximately 110° around the circumference of the inner tube 14 and thus provides the surgeon greater access into the spinal canal. The opening 16 may be formed with a pair of tracks 44 on its longitudinally extending walls to receive shutter 18. Preferably, shutter 18 as mounted within the inner tube 14 is approximately flush with the exterior surface of the inner tube. As shown in FIGS. 1 and 3, a second worm drive 50 is mounted to a second snap ring 52 that is received on the upper or proximal edge of the inner tube 14. The second worm drive 50 mates with teeth 54 formed on a side surface of the shutter 18. The second worm drive is approximately 52 mm in length. As the second worm drive 50 is rotationally twisted in one direction at an upper surface of the outer tube, the shutter is extended over a greater portion of the window. Likewise, when the second worm drive 50 is rotationally twisted in the opposite directions, the shutter 18 retracts away from the window 16 thereby providing greater access to the spinal canal. The surgeon can also adjust the medial window using the scale on the shutter.

The shutter 18 may be extended beyond the distal end 42 of the inner tube such that the shutter extends into the spinal canal. This provides the surgeon with the ability to restrain additional tissue within the surgical field.

Preferably, the outer tube diameter is approximately 28 mm and the inner diameter is approximately 19.5 mm. It is to be understood that the dimensions provided in this disclosure could vary from these without departing from the scope of the invention.

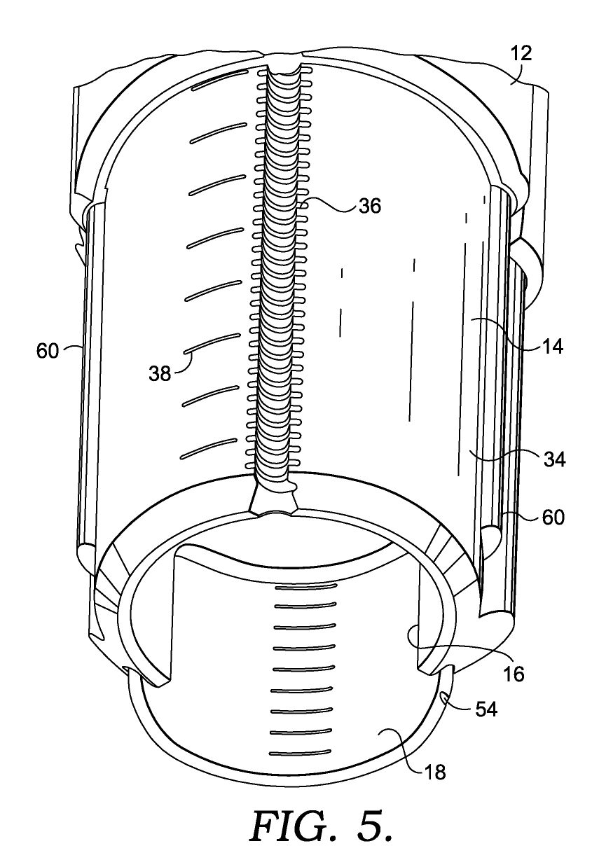

Identifying anatomical landmarks is paramount to surgical success. To this end, superior and inferior endoscope ports 60 are provided on the access device as shown in FIGS. 4 and 5 to provide a protected and constrained environment for two 1.5 mm endoscopes. Another option is to use one endoscope port for an additional light source and the other for an endoscope.

What is claimed is:

1. An access device for insertion into a patient through an incision to a spinal location, comprising: an elongate body having an outer tube and an inner tube, a proximal end of the outer tube and a distal end of the inner tube defining a first length therebetween such that the proximal end can be positioned outside the patient and the distal end can be positioned inside the patient through the incision adjacent the spinal location; the elongate body having an outer surface and an inner surface defining a passage, the elongate body further comprising a first drive for selectively extending and retracting the inner tube relative to the outer tube to a length other than the first length.

2. An access device for insertion into a patient through an incision to a spinal location, comprising: an elongate body having an outer tube and an inner tube, a proximal end of the outer tube and a distal end of the inner tube defining a first length therebetween such that the proximal end can be positioned outside the patient and the distal end can be positioned inside the patient through the incision adjacent the spinal location; the outer tube and inner tube each having an outer surface and an inner surface, the inner surface of the outer and inner tube defining a passage for the elongate body, the inner tube further comprising an opening extending along a periphery of the inner tube and a selectively movable shutter mounted to the inner tube and configured to cover a portion of the opening.

3. A method for accessing a spinal location inside a patient, the method comprising the steps of:
creating an incision in the patient for a desired spinal location;
providing an access device having an outer tube and an inner tube, the inner tube being telescopically received in the outer tube and selectively movable relative to the outer tube through a drive,
selecting a length for the access device prior to insertion into the patient,
inserting the access device, and
adjusting the length of the access device while the inner tube is at least partially inside the patient to a length that optimizes a working space for a surgeon.

4. A method for accessing a spinal location inside a patient, the method comprising the steps of:
creating an incision in the patient for a desired spinal location;
providing an access device having an outer tube and an inner tube, the inner tube being telescopically received in the outer tube and selectively movable relative to the outer tube through a drive, the inner tube further being configured with an opening around a periphery of the inner tube adjacent a distal end of the inner tube to define a window, the access device further comprising a selectively movable shutter mounted to the inner tube in the window for selective covering a portion of the window;
selecting a length for the access device prior to insertion into the patient,
inserting the access device,
adjusting the length of the access device while the inner tube is at least partially inside the patient to a length that optimizes a working space for a surgeon; and
adjusting the shutter to selectively open a portion of the window to further optimize the working space for the surgeon.
